(19) United States
(12) Patent Application Publication
(10) Pub. No.: US 2010/0068306 A1
(43) Pub. Date: Mar. 18, 2010

(54) COMPOSITIONS FOR ACCELERATING THE HEALING RESPONSE

(76) Inventor: Chin-Pao Chou, Shafter, CA (US)

Correspondence Address: Jennifer M. Chou, 28549 West Tulare Ave., Shaffer, CA, 93263, US

(21) Appl. No.: 12/559,273

(22) Filed: Sep. 14, 2009

Related U.S. Application Data
(60) Provisional application No. 61/192,260, filed on Sep. 16, 2008.

(51) Int. Cl. A61K 33/06; A61P 31/00

(52) U.S. Cl. 424/698

(57) ABSTRACT

An herbal composition capable of helping to speed the body's healing reaction to infection, and methods of treating wounds and infections using such a composition are provided. The composition provides an herbal medicine combining Rhizome of Chinese Goldthread, Indigo Naturalis, Menthae Arvensis, Alum, Bornolum Syntheticum, Catechu and Licorice, for external use, which can be applied topically to encourage the healing effect of a body and prevent or eliminate infection

### CROSS-REFERENCE TO RELATED APPLICATIONS

This application claims priority to U.S. Provisional Application No. 61/192,260, filed Sep. 16, 2008, the disclosure of which is incorporated herein by reference.

### FIELD OF THE INVENTION

This invention relates generally to a novel composition for accelerating the body's natural healing response; and more particularly to a composition comprising a combination of herbs and minerals for external application capable of helping to ameliorate the effects of infection.

### BACKGROUND OF THE INVENTION

Traditional Chinese medicine is an ancient healing system originating almost 4,500 years ago. It continues to be the most common healing system in China today despite the strong presence of Western Medicine.

The first Chinese manual on pharmacology, the Shennong Bencao Jing (Shennong Emperor's Classic of Materia Medica), lists some 365 medicines of which 252 of them are herbs, and dates back somewhere in the 1st century C. E. Han dynasty. Succeeding generations of herbalists have augmented on this work, as in the Yaoxing Lun; literally “Treatise on the Nature of Medicinal Herbs”), a 7th century Tang Dynasty Chinese treatise on herbal medicine, and the Compendium of Materia Medica (Bencao Gangmu) compiled during the Ming dynasty by Li Shizhen, which is still used today for consultation and reference. The history of this literature is presented in Paul U. Unschuld's “Medicine in China: a History of Pharmaceutics”; Univ. of Calif. Press, 1986. The Shen Nong's Herbal Classic, a 2000-year old medicinal Chinese book considered today as the oldest book on oriental herbal medicine.

Chinese medicine has a very unique approach to its study of the human body and the interrelationships of the various body systems. For example, Shennong classifies 365 species of roots, grass, woods, furs, animals and stones into three categories of herbal medicine:
The first category, called “superior”, includes herbs effective for multiple diseases and are mostly responsible for maintaining and restoring the body balance. They have almost no unfavorable side-effects.
The second category comprises tonics and boosters, for which their consumption must not be prolonged.
The third category must be taken, usually in small doses, and for the treatment of specific ailments only.

Many aspects of Chinese medicine are now practiced widely in North America in many forms including, acupuncture, acupressure, moxabustion, and dispensing of Chinese herbal formulas. In fact, many Western herbalists are now incorporating Chinese herbs into their formulas as well. In particular, the ancient Chinese use of medicinal mushrooms has inspired modern day research into mushrooms like shiitake, Agaricus blazei, Trametes versicolor, and of course lingzhi. A 2008 Review, by U C Davis, stressed the urgency of further research and future clinical trials into the use of mushrooms or mushroom extracts in the treatment of disease due to large numbers of promising in vivo and in vitro experiments.

However, currently the research into the effectiveness of these Chinese herbs has not extended far past these mushroom extracts. Accordingly, a need exists to find novel compositions that can capitalize on the demonstrated effectiveness of Chinese herbs to ameliorate the effects of infection on the human body.

### BRIEF SUMMARY OF THE INVENTION

Thus, there is provided in accordance with the current invention an herbal composition for topical application capable of ameliorating the effects of infection and improving the body's healing response.

In one embodiment, the invention is directed to a composition for use in treating an external injury of the body, comprising at least the following active ingredients: Rhizome of Chinese Goldthread, Indigo Naturalis, Menthae Arvensis, Alum, Bornolum Syntheticum, Catechu, and Licorice.

In another embodiment, the composition of the invention has a concentration of Rhizome of Chinese Goldthread of from 10 to 35% by volume of the composition.

In still another embodiment, the composition of the invention has a concentration of Indigo Naturalis of from 10 to 35% by volume of the composition.

In yet another embodiment, the composition of the invention has a concentration of Menthae Arvensis of at least 1% by volume of the composition.

In still yet another embodiment, the composition of the invention has a concentration of Alum of at least 1% by volume of the composition.

In still yet another embodiment, the composition of the invention has a concentration of Bornolum Syntheticum of at least 1% by volume of the composition.

In still yet another embodiment, the composition of the invention has a concentration of Catechu of at least 1% by volume of the composition.

In still yet another embodiment, the composition of the invention has a concentration of Licorice of at least 10% by volume of the composition.

In still yet another embodiment, the composition of the invention has the following formulation: 27% Rhizome of Chinese Goldthread, 23% Indigo Naturalis, 10% Menthae Arvensis, 10% Alum, 10% Bornolum Syntheticum, 10% Catechu, and 10% Licorice each by volume.

In still yet another embodiment, the composition takes the form of a topical medicinal selected from the group selected from the list consisting of creams, ointments, lotions, gels, balms, patches, pastes, spray solutions and aerosols.

In still yet another embodiment, the composition further comprises at least one non-active ingredient selected from the group consisting of solvents, preservatives, emulsifying agents, suspending agents, diluents, scents and coloring agents.

In still yet another embodiment, the composition comprises from 0.01 to 95% by volume of the total topical medicinal composition.

In another embodiment, the current invention is directed to a method of treating an external injury to the body of a patient comprising administering a composition having the following active ingredients: Rhizome of Chinese Goldthread, Indigo Naturalis, Menthae Arvensis, Alum, Bornolum Syntheticum, Catechu, and Licorice.

### DETAILED DESCRIPTION OF THE INVENTION

The current invention is directed to an herbal composition capable of helping to speed the body's healing reaction to infection. In particular, the current invention recognizes that infection results in areas of the body being overly heated, which can result in additional damage to the cells of the infected area. Accordingly, the current invention provides an herbal medicine for external use comprised of a combination of six herbs and one mineral, which can be applied topically and has the distinct character of providing a cooling effect to the body, thereby encouraging the healing of the body and the prevent or eliminate infection.

In general terms, the several herbal and mineral components of the current invention include the following:
Rhizome of Chinese Goldthread, which is included for its antibacterial and anti-infection qualities;
Indigo Naturalis, which is included for its general cooling and anti-infection, qualities;
Menthae Arvesis, which is included for its anti-inflammatory properties, namely its action to cause contraction of the capillaries and mucus membranes;
Alum, which is included for its general anti-toxin and anti-parasite properties, and its beneficial impact on the health of the flesh;
Borneolum Syntheticum, which also has cooling properties and its ability to wick away heat and promote tissue regeneration; Catechu, which is a powerful mucilaginous juice that can promote effective healing of wounds; and
Licorice, which is a demulcent to irritated membranes, an expectorant that can help in the loosening and expulsion of congestion, and which is traditionally used to enhance the flavor and effectiveness of herbal compositions.

Turning to each of the components in detail, Rhizome of Chinese Goldthread is taken from the underground stem of Cotis Chinensis French, a perennial herb of the family Ranunculaceae. Rhizome of Chinese Goldthread produces a mass of thread-like golden rhizomes that may be used in healing. In traditional Chinese medicine this herb is used mainly to improve digestive function and to prevent the growth of microorganisms. It has a bitter flavor, and its action is to cool or purge intense heat, eliminate dampness, and remove toxic substances.

Indigo Naturalis, Isatic Incligotica Fort. Extract, is a dried powder prepared from the leaf of the stem of the plant Baphicaclanthus Cusua (Ness) Bremek .Fam Acantheceae Polygonum Thintorium Ait. Istasis Indigotica Fort .Fam Cruciferae. Indigo is a deep blue powder, which is generally ground into a fine powder. It has a sally flavor, and its action is generally cooling, and to clear heat and remove toxicity. In particular, indigo is known to have the effect of treating abnormal heating of the blood.

Borneolum Syntheticum is produced from the plant Dryobalanops Aromatic Gaertn. F, of the family Dipterocarpceae. The medicinal material is made from the leaf of Artemisia Argyi Levl. Et Vent. The herb may also be synthesized from a camphor and turpentine oil mixture that is marketed under the name Synthetic One. The finished product is ground into powder. It is pungent of smell and bitter of flavor. Again, Borneolum Syntheticum has the action of clearing away heat and alleviating pain.

Catechu or Acacia Catechu is a small to medium size thorny tree with a dark grey or grayish brown bark. It has indodorous properties and has an astringent/bitter taste followed by a light sweetness. Its typical medicinal form is as Kersal, which is a crystalline form of Catechu sometimes found deposited in the cavities of the wood. Kersal is used medicinally to treat coughs and sore throats. The tree also has a powerfully protective mucilaginous juice. One of the remarkable properties of this juice is its ability to resist fire while retaining its water content. As a result, this juice is often used to treat or resist injury from heat or fire.

Methae Arvensis, also known as menthe pipenta or bo he mint is a wild mint that is acrid and has cooling properties. In particular, Methae Arvensis combines methos and methal derivatives, which help promote secretions and rid the body of mucous that attaches to membranes, and which possesses the substance cholagogue, and which is known to promote bile secretions. It also contains essential oils. The application of this mint medication is known to make the skin feel cool, and causes the capillaries of the mucus to contract. It has been shown to result in an anti-inflammatory effect, and arrest pain and relieve itchiness.

Alum is a processed mineral of sulfates of the alum stone. It mainly contains potassium aluminum sulfate. It is an astringent that has a very sour taste. Its typical actions are to counteract toxicity, to kill parasites, and to relieve itching and arrest bleeding.

Licorice Glycyrrhiza is a root having an extremely sweet taste with bitter after-tones. Licorice is generally thought to neutralize toxins, relieve inflammation, and it is considered a demulcent (i.e., a substance soothing to irritated membranes). Among its well-documented active effects include its use as an anti-inflammatory. It is also used in traditional Chinese medicine to treat hepatitis, sore throat and spasms. And, it is also used in Chinese herb prescriptions to enhance the effectiveness of formulas, and to moderate the flavor of teas and other herbs.

Although these ingredients can be combined in many different proportions, in one preferred embodiment, the composition contains the following operative ranges and, likewise, the preferred composition:

TABLE 1
Compositional Ranges

| | Operative | Preferred |
|---|---|---|
| Component | Range (vol.) | Composition (vol.) |
| Rhizome of Chinese Goldthread | 10 to 35% | 27% |
| Indigo Naturalis | 10 to 35% | 23% |
| Menthae Arvensis | at least 1% | 10% |
| Alum | at least 1% | 10% |
| Bornolum Syntheticum | at least 1% | 10% |
| Catechu | at least 1% | 10% |
| Licorice | at least 10% | 10% |

The compositions according to the invention can be made by any suitable method known to those skilled in the art. For example, the various herbs and minerals of the composition can be obtained or processed and then ground into the form of a powder. For relatively small batches (e.g., less than 10 kilos), the desired amount of each component may be measured and put into a large plastic bag. The plastic bag is then sealed with a large amount of air remaining in the bag. The bag can then be shaken and tossed for about ten minutes to thoroughly mix the composition. For larger batches, the components can be mixed in a large stainless steel mixer, or other suitable industrial processing machine.

The blended composition can then be provided in any suitable external dosage form, such as a cream, ointment, lotion, gel, balm, patch, paste, spray solution, aerosol, etc. Further, any suitable non-active ingredients necessary to allow for the creation of a suitable external dose may be used with the composition of the current invention. For example, if a simple rub is required the composition may be mixed with water or some other suitable liquid medium and a paste formed. Alternatively, if a topical ointment dosage form is desired, the composition can be mixed with aqueous and nonaqueous solutions, emulsions, suspensions and solutions and/or suspensions reconstituted from non-effervescent granules, containing suitable solvents, preservatives, emulsifying agents, suspending agents, diluents, scents and coloring agents. The active ingredients should be included in the final topical medicine in an amount of from 0.01 to 95% by volume of the topical solution.

The compositions can be administered to a patient by any suitable method, and are preferably administered topically. As used herein, the term “patient” refers to a human being. A preferred dosage for adult humans is a sufficient amount to cover the affected area. The composition should be reapplied as necessary to ensure that the affected area is completely covered until the symptoms recede.

### Exemplary Embodiments

The person skilled in the art will recognize that additional embodiments according to the invention are contemplated as being within the scope of the foregoing generic disclosure, and no disclaimer is in any way intended by the foregoing, non-limiting examples. It should be understood that the composition of the current invention has been exhaustively tested on numerous subjects with the result that wounds or infections from wounds are healed with a result, both in terms of speed of healing and final outcome, far superior to untreated patients.

### Example 1

### Treatment of a Typical Cut

A composition containing 27% by volume of Rhizome of Chinese Goldthread, 23% of Indigo Naturalis, 10% of Menthae Arvensis, 10% of Alum, 10% of Bornolum Syntheticum, 10% of Catechu and 10% of Licorice, each by volume, is ground into a powder and mixed together with water to form a paste. The paste is then administered to a patient having a cut that is bleeding. The patient would be instructed to immediately place enough of the composition to cover the entire wounded area, and reapply as needed to ensure proper coverage until the wound is healed and the risk of infection has passed. It has been observed that such a use of the composition prevents the development of infection around the wound, and also improves the healing time of the wound by several days over an untreated wound.

### Example 2

### Treatment of a Typical Skin Infection

A patient having an insect bite where there is a noticeable ring of discoloration around the bite indicating irritation or infection would mix the composition as described in Example 1, above. This paste would then be applied to the insect bite and the whole infected and discolored area, and reapplied as needed to ensure proper coverage until the infection is gone or until it helps to regenerate healthy flesh. Again, it has been observed that by liberal application of the inventive compound to such irritated or infected wounds that the infection can be resolved and the wound healed at a substantially accelerated rate over untreated bites.

### Doctrine of Equivalents

Those skilled in the art will appreciate that the foregoing examples and descriptions of various preferred embodiments of the present invention are merely illustrative of the invention as a whole, and that variations in the steps and various components of the present invention may be made within the spirit and scope of the invention. For example, it will be clear to one skilled in the art that additional inactive ingredients or insubstantial amounts of other active ingredients would not affect the improved properties of the composition of the current invention nor render it unsuitable for its intended purpose. Accordingly, the present invention is not limited to the specific embodiments described herein but, rather, is defined by the scope of the appended claims.

What is claimed is:

1. A composition for use in treating an external injury of the body, comprising at least the following active ingredients: Rhizome of Chinese Goldthread, Indigo Naturalis, Menthae Arvensis, Alum, Bornolum Syntheticum, Catechu, and Licorice.

2. The composition of claim 1, wherein the Rhizome of Chinese Goldthread comprises from 10 to 35% by volume of the composition.

3. The composition of claim 1, wherein the Indigo Naturalis comprises from 10 to 35% by volume of the composition.

4. The composition of claim 1, wherein the Menthae Arvensis comprises at least 1% by volume of the composition.

5. The composition of claim 1, wherein the Alum comprises at least 1% by volume of the composition.

6. The composition of claim 1, wherein the Bornolum Syntheticum comprises from 1% by volume of the composition.

7. The composition of claim 1, wherein the Catechu comprises at least 1% by volume of the composition.

8. The composition of claim 1, wherein Licorice comprises at least 10% by volume of the composition.

9. The composition of claim 1, wherein the composition has the following formulation 27% Rhizome of Chinese Goldthread, 23% Indigo Naturalis, 10% Menthae Arvensis, 10% Alum, 10% Bornolum Syntheticum, 10% Catechu, and 10% Licorice each by volume.

10. The composition of claim 1 wherein the composition is made into a topical medicinal selected from the group selected from the list consisting of creams, ointments, lotions, gels, balms, patches, pastes, spray solutions and aerosols.

11. The composition of claim 10, wherein the composition further comprises at least one non-active ingredient selected from the group consisting of solvents, preservatives, emulsifying agents, suspending agents, diluents, scents and coloring agents.

12. The composition of claim 10, wherein the active ingredient should comprise from 0.01 to 95% by volume of the total topical medicinal composition.

13. A method of treating an external injury to the body of a patient comprising:
providing a composition comprising at least the following active ingredients: Rhizome of Chinese Goldthread, Indigo Naturalis, Menthae Arvensis, Alum, Bornolum Syntheticum, Catechu, and Licorice;
administering said composition topically to said external injury; and
readministering said composition as necessary until said injury is healed.

14. The method of claim 1, wherein the Rhizome of Chinese Goldthread comprises from 10 to 35% by volume of the composition.

15. The method of claim 13, wherein the Indigo Naturalis comprises from 10 to 35% by volume of the composition.

16. The method of claim 13, wherein the Menthae Arvensis comprises at least 1% by volume of the composition.

17. The method of claim 13, wherein the Alum comprises at least 1% by volume of the composition.

18. The method of claim 13, wherein the Bornolum Syntheticum comprises from 1% by volume of the composition.

19. The method of claim 13, wherein the Catechu comprises at least 1% by volume of the composition.

20. The method of claim 13, wherein Licorice comprises at least 10% by volume of the composition.

21. The method of claim 13, wherein the composition has the following formulation 27% Rhizome of Chinese Goldthread, 23% Indigo Naturalis, 10% Menthae Arvensis, 10% Alum, 10% Bornolum Syntheticum, 10% Catechu, and 10% Licorice each by volume.

22. The method of claim 13 wherein the composition is a topical medicinal selected from the group selected from the list consisting of creams, ointments, lotions, gels, balms, patches, pastes, spray solutions and aerosols.

23. The method of claim 22, wherein the composition further comprises at least one non-active ingredient selected from the group consisting of solvents, preservatives, emulsifying agents, suspending agents, diluents, scents and coloring agents.

24. The method of claim 22, wherein the active ingredient should comprise from 0.01 to 95% by volume of the total topical medicinal composition.
